(19) United States
(12) Patent Application Publication
(10) Pub. No.: US 2018/0170834 A1
(43) Pub. Date: Jun. 21, 2018

(54) REMOVAL OF FEED TREATMENT UNITS IN AROMATICS COMPLEX DESIGNS

(71) Applicant: UOP LLC, Des Plaines, IL (US)

(72) Inventors: Kathryn R. Bjorkman, Oak Park, IL (US); Patrick C. Whitchurch, Sleepy Hollow, IL (US); Rodrigo J. Lobo, Des Plaines, IL (US); Linda S. Cheng, Highland Park, IL (US); Susan A. Somers, Elmhurst, IL (US)

(21) Appl. No.: 15/846,331

(22) Filed: Dec. 19, 2017

Related U.S. Application Data
(60) Provisional application No. 62/436,916, filed on Dec. 20, 2016.

(51) Int. Cl. C07C 5/27; C07C 15/08; C07C 7/13; C07C 7/00; B01J 20/18; B01J 20/28; B01D 3/14; B01D 3/42; B01D 53/08; C10G 35/04

(52) U.S. Cl. CPC C07C 5/2702; C07C 15/08; C07C 7/13; C07C 7/005; B01J 20/186; B01D 2253/108; B01D 3/14; B01D 3/4283; B01D 53/08; C10G 35/04; B01J 20/28007

(57) ABSTRACT

Processes and apparatuses for producing para-xylenes are provided. The processes comprises providing a reformate stream comprising aromatic hydrocarbons to a reformate splitter to provide a reformate bottoms stream and a reformate overhead stream. A portion of the reformate bottoms stream is passed to a para-xylene separation unit for separating para-xylene, wherein the portion of the reformate bottoms stream is passed to the para-xyelene separation unit without an intermediate step for removal of olefins.

### CROSS-REFERENCE TO RELATED APPLICATION

This application claims priority from Provisional Application No. 62/436,916 filed Dec. 20, 2016, the contents of which cited application are hereby incorporated by reference in its entirety.

### TECHNICAL FIELD

The technical field generally relates to apparatuses and processes for producing xylene isomers in an aromatics complex. More particularly, the present disclosure relates to removal of olefin removal units in an aromatics complex producing para-xylene.

### BACKGROUND

Most new aromatics complexes are designed to maximize the yield of benzene and C₈ aromatic isomer (para-xylene, meta-xylene, ethylbenzene and ortho-xylene). Para-xylene, meta-xylene and ortho-xylene, are important intermediates which find wide and varied application in chemical syntheses. Para-xylene upon oxidation yields terephthalic acid which is used in the manufacture of synthetic textile fibers and resins. Meta-xylene is used in the manufacture of plasticizers, azo dyes, wood preservers, etc. Ortho-xylene is feedstock for phthalic anhydride production. The distribution of xylene isomers from catalytic reforming and other sources generally does not match that of the sought isomers for chemical intermediates and thus the producer converts the feedstocks to generate more of the sought isomers in the aromatics complexes.

The production of xylenes is practiced commercially in large-scale facilities and is highly competitive. Concerns exist not only about the effective conversion of feedstock through one or more of isomerization, transalkylation and disproportionation to product xylenes, but also other competitive aspects with respect to such facilities including capital costs and energy costs.

A prior art aromatics complex flow scheme has been disclosed by Meyers in the Handbook of Petroleum Refining Processes, 2d. Edition in 1997 by McGraw-Hill.

Various sources have been proposed for monocyclic aromatics as a feed to a xylene production facility. The most prevalent are the catalytic reforming of naphtha fractions and pyrolysis followed by hydrotreating of naphtha fractions. These processes typically produce a wide spectrum of chemical compounds including not only the sought monocyclic aromatics but also polycyclic aromatics and olefins. Polycyclic aromatics and olefins are typically undesirable impurities in xylene production facilities. They can have a negative impact on the product quality and the efficiency of the processes such as by requiring additional process steps, reducing catalyst life, decreasing stability of the product, and causing undesirable product color. Polycyclic aromatics are typically removed by distillation from the desired monocyclic aromatics. These removed polycyclic aromatics are then disposed of in any suitable manner, usually as a fuel, and thus have lesser value. It is also known that the polycyclic aromatics can be converted to useful monocyclic aromatics such as toluene, xylenes and C₉₊ monocyclic aromatics.

The quality of feed streams to the various process units within a xylene production facility is also specified to ensure proper performance. For example, the olefin content of streams fed to some process units of aromatics complex, including the para-xylene separation unit, is limited. Thus, olefin are recognized as a contaminant in the feed to the adsorbent present in the para-xylene separation unit and the conventional practice is to reduce the olefin content to an acceptable level (feed specification limit) using various olefin removal processes such as hydrotreating, hydrogenation, treating with clay and/or molecular sieves and olefin reduction process (ORP). Olefins are for example commonly removed from the xylene production facility feedstock and/or intermediate streams at various locations within the facility by clay treating. In clay treaters, olefins are converted to oligomers which can cause fouling of the clay. The cost to operate clay treaters, including reloading them with fresh clay and disposal of the organic contaminated spent clay, can be a significant financial burden on the commercial-scale producer of xylenes. Moreover, clay treaters can result in alkylation of an olefin to an aromatic ring. Hence, the effluent from a clay treater can contain aromatic rings having C₂₊ substituents such as ethylbenzene, propylbenzene, and methylethylbenzene. Thus the value of the aromatic feedstock for the production of benzene, toluene and xylene is reduced.

Accordingly, it is desirable to provide an improved and cost-effective process and apparatus for production of xylene isomers. Furthermore, other desirable features and characteristics of the present subject matter will become apparent from the subsequent detailed description of the subject matter and the appended claims, taken in conjunction with the accompanying drawings and this background of the subject matter.

### BRIEF SUMMARY

Various embodiments contemplated herein relate to apparatuses and processes for producing xylene isomers in an aromatics complex. The exemplary embodiments taught herein illustrate removal of olefin removal units between one or more process units present in an aromatics complex producing para-xylenes.

In accordance with another exemplary embodiment, a process is provided for the production of para-xylene comprising providing a reformate stream comprising aromatic hydrocarbons to a reformate splitter to provide a reformate bottoms stream comprising C₇₊ aromatic hydrocarbons and a reformate overhead stream comprising C₇₋ aromatic hydrocarbons. A portion of the reformate bottoms stream is passed to a para-xylene separation unit for separating para-xylene, wherein the portion is contacted with an adsorbent under adsorption conditions to provide a xylene extract stream comprising para-xylene and a raffinate product stream, wherein the portion of the reformate bottoms stream is passed to the para-xyelene separation unit without an intermediate step for removal of olefins.

In accordance with another exemplary embodiment, a process is provided for the production of para-xylene comprising introducing a raffinate product stream comprising C₈ aromatic isomers to an isomerization unit to provide an isomerization effluent, wherein the isomerization effluent is produced in the presence of an ethylbenzene (EB) isomerization catalyst. The isomerization effluent is passed to a deheptanizer column to provide a deheptanizer bottoms stream comprising C₇₊ aromatics. A portion of the deheptanizer bottoms stream is passed to a para-xylene separation unit for separating para-xylene, wherein the portion is contacted with an adsorbent under adsorption conditions to provide a xylene extract stream comprising para-xylene and the raffinate product stream, wherein the portion of the deheptanizer bottoms stream is passed to the para-xyelene separation unit without an intermediate step for removal of olefins.

In accordance with yet another exemplary embodiment, a process is provided for the production of para-xylene comprising providing a reformate stream comprising aromatic hydrocarbons to a reformate splitter to provide a reformate bottoms stream comprising C₇₊ aromatic hydrocarbons and a reformate overhead stream comprising C₇₋ aromatic hydrocarbons. A raffinate product stream comprising C₈ aromatic isomers is introduced to an isomerization unit to provide an isomerization effluent, wherein the isomerization effluent is produced in the presence of an ethylbenzene (EB) isomerization catalyst. The isomerization effluent is passed to a deheptanizer column to provide a deheptanizer bottoms stream comprising C₇₊ aromatics. A portion of the reformate bottoms stream and a portion of the deheptanizer bottoms stream are passed to a para-xylene separation unit for separating para-xylene, wherein the reformate bottom portion and the deheptanizer bottom portion are contacted with an adsorbent under adsorption conditions to provide a xylene extract stream comprising para-xylene and the raffinate product stream, wherein the portion of the isomerization effluent and the portion of the deheptanizer bottoms stream is passed to the para-xyelene separation unit without an intermediate step for removal of olefins.

These and other features, aspects, and advantages of the present disclosure will become better understood upon consideration of the following detailed description, drawings and appended claims.

### BRIEF DESCRIPTION OF THE DRAWINGS

The various embodiments will hereinafter be described in conjunction with the following FIGURES, wherein like numerals denote like elements.

FIG. 1 illustrates an aromatics complex according to an embodiment of the present disclosure.

FIG. 2 illustrates an aromatics complex according to another embodiment of the present disclosure.

Corresponding reference characters indicate corresponding components throughout the several views of the drawings. Skilled artisans will appreciate that elements in the figures are illustrated for simplicity and clarity and have not necessarily been drawn to scale. For example, the dimensions of some of the elements in the figures may be exaggerated relative to other elements to help to improve understanding of various embodiments of the present disclosure. Also, common but well-understood elements that are useful or necessary in a commercially feasible embodiment are often not depicted in order to facilitate a less obstructed view of these various embodiments of the present disclosure.

### Definitions

As used herein, the term “stream” can include various hydrocarbon molecules and other substances.

As used herein, the term “stream”, “feed”, “product”, “part” or “portion” can include various hydrocarbon molecules, such as straight-chain and branched alkanes, naphthenes, alkenes, alkadienes, and alkynes, and optionally other substances, such as gases, e.g., hydrogen, or impurities, such as heavy metals, and sulfur and nitrogen compounds. Each of the above may also include aromatic and non-aromatic hydrocarbons.

As used herein, the term “overhead stream” can mean a stream withdrawn at or near a top of a vessel, such as a column.

As used herein, the term “bottoms stream” can mean a stream withdrawn at or near a bottom of a vessel, such as a column.

Hydrocarbon molecules may be abbreviated C₁, C₂, C₃, Cn where “n” represents the number of carbon atoms in the one or more hydrocarbon molecules or the abbreviation may be used as an adjective for, e.g., non-aromatics or compounds. Similarly, aromatic compounds may be abbreviated A₆, A₇, A₈, An where “n” represents the number of carbon atoms in the one or more aromatic molecules. Furthermore, a superscript “+” or “−” may be used with an abbreviated one or more hydrocarbons notation, e.g., C₃₊ or C₃₋, which is inclusive of the abbreviated one or more hydrocarbons. As an example, the abbreviation “C₃₊” means one or more hydrocarbon molecules of three or more carbon atoms.

As used herein, the term “zone” or “unit” can refer to an area including one or more equipment items and/or one or more sub-zones. Equipment items can include, but are not limited to, one or more reactors or reactor vessels, separation vessels, distillation towers, heaters, exchangers, pipes, pumps, compressors, and controllers. Additionally, an equipment item, such as a reactor, dryer, or vessel, can further include one or more zones or sub-zones.

The term “column” means a distillation column or columns for separating one or more components of different volatilities. Unless otherwise indicated, each column includes a condenser on an overhead of the column to condense and reflux a portion of an overhead stream back to the top of the column and a reboiler at a bottom of the column to vaporize and send a portion of a bottoms stream back to the bottom of the column. Feeds to the columns may be preheated. The top or overhead pressure is the pressure of the overhead vapor at the vapor outlet of the column. The bottom temperature is the liquid bottom outlet temperature. Overhead lines and bottoms lines refer to the net lines from the column downstream of any reflux or reboil to the column unless otherwise shown. Stripping columns omit a reboiler at a bottom of the column and instead provide heating requirements and separation impetus from a fluidized inert media such as steam.

As used herein, the term “rich” can mean an amount of at least generally 50%, and preferably 70%, by mole, of a compound or class of compounds in a stream.

As depicted, process flow lines in the FIGURES can be referred to interchangeably as, e.g., lines, pipes, feeds, gases, products, discharges, parts, portions, or streams.

The term “communication” means that material flow is operatively permitted between enumerated components.

The term “downstream communication” means that at least a portion of material flowing to the subject in downstream communication may operatively flow from the object with which it communicates.

The term “upstream communication” means that at least a portion of the material flowing from the subject in upstream communication may operatively flow to the object with which it communicates.

The term “direct communication” means that flow from the upstream component enters the downstream component without undergoing a compositional change due to physical fractionation or chemical conversion.

The term “predominantly” means a majority, suitably at least 50 mol % and preferably at least 60 mol %.

The term “passing” means that the material passes from a conduit or vessel to an object.

The term “majority” means, suitably at least 40 wt % and preferably at least 50 wt %.

### DETAILED DESCRIPTION

The following detailed description is merely exemplary in nature and is not intended to limit the various embodiments or the application and uses thereof. Furthermore, there is no intention to be bound by any theory presented in the preceding background or the following detailed description. Moreover, the reaction conditions including selection of temperature, pressure, LHSV and catalyst in the various units in the aromatics complex described below are conventional which are known to one of ordinary skill in the art, unless wherever mentioned.

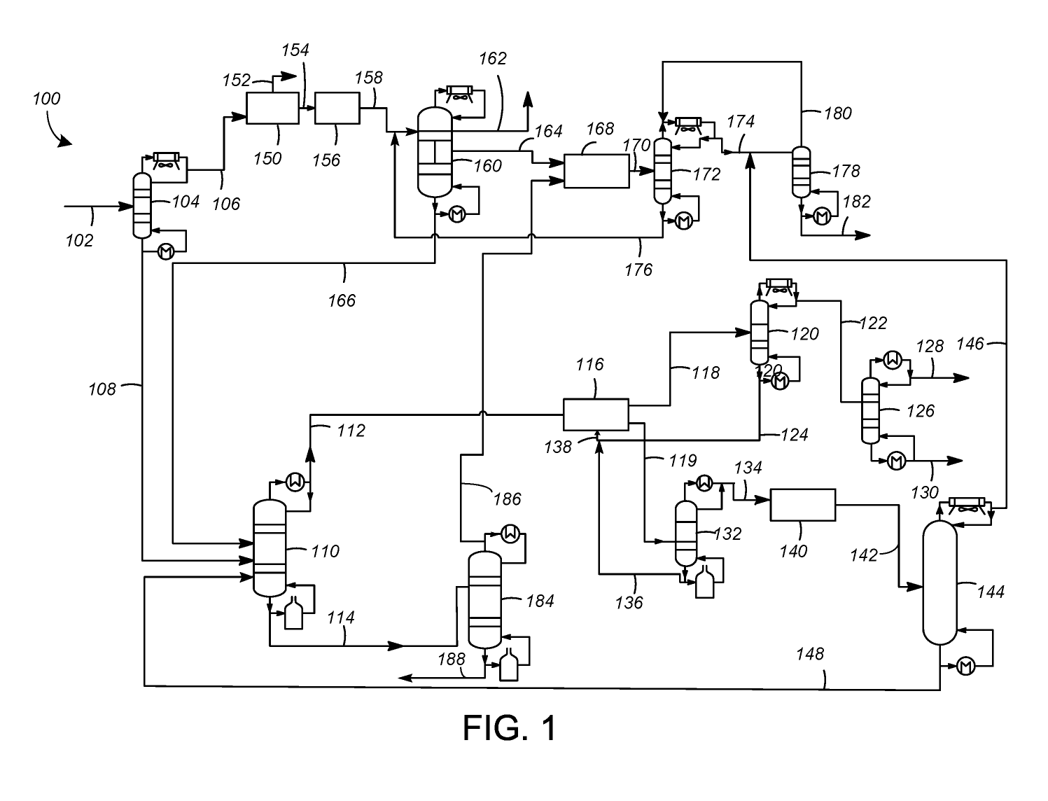

Various embodiments are directed to apparatuses and processes for producing a C₈ aromatic isomer product in an aromatic complex. An exemplary embodiment of the process and apparatus for producing a para-xylene product in an aromatic complex is addressed with reference to a process and apparatus 100 illustrating an aromatics complex according to an embodiment as shown in FIG. 1. The process and apparatus 100 includes a reformate splitter column 104, a xylene fractionation column 110, a para-xylene separation unit 116, an extract column 120, a finishing column 126, a raffinate column 132, an isomerization unit 140, a deheptanizer column 144, an aromatics extraction unit 152, a benzene-toluene (BT) column 160, a transalkylation unit 168, a transalkylation stripper 172, a stabilizer 178 and a heavy aromatics column 184.

In accordance with an exemplary embodiment as shown in FIG. 1, a reformate stream in line 102 comprising aromatic hydrocarbons may be passed to the reformate splitter column 104. A reformate overhead stream in line 106 comprising C₇₋ aromatic hydrocarbons and a reformate bottoms stream in line 108 comprising C₇₊ aromatic hydrocarbons may be withdrawn from the reformate splitter column 104. In accordance with an instant embodiment as shown, an overhead stream from the reformate splitter column 106 may be condensed and separated in a receiver with a portion of the condensed liquid being refluxed back to the reformate splitter column 104 to obtain the reformate overhead stream from a net portion in line 106. Further, as illustrated, the reformate splitter column 104 may include a reboiler at a bottom of the column to vaporize and send a portion of the reformate bottoms stream back to the bottom of the column. A portion of the reformate bottoms stream may be passed to the para-xylene separation unit 116 for separating para-xylene, described in detail later. The portion of the reformate bottoms stream may be passed to the para-xylene separation unit 116 without an intermediate step for removal of olefins. Accordingly, there is no intermediate treatment unit. In accordance with an exemplary embodiment as shown in the FIG. 1, the reformate bottoms stream in line 108 may be passed to the xylene fractionation column 110 for separation. Typically, the reformate bottoms stream in line 108 is passed through an olefin treatment unit to treat residual olefin contaminants before being passed to the xylene fractionation column 110. Examples of the olefin treatment unit include, but are not limited to, a clay treater and an olefin reduction process (ORP) unit. In accordance with an exemplary embodiment as shown in FIG. 1, the reformate bottoms stream in line 108 may be passed directly to the xylene fractionation column 110 without an intermediate step for removal of olefins. Accordingly, the xylene fractionation column 110 may be in direct, downstream communication with the reformate splitter column 104. The xylene fractionation column 110 may further receive a benzene toluene (hereinafter “BT”) column bottoms stream in line 166 comprising xylenes. Further, the xylene fractionation column 110 may receive C₈₊ aromatic hydrocarbons in a deheptanizer bottoms stream in line 148 from the deheptanizer column 144. In embodiments, there can be more than xylene fractionation columns. In accordance with an exemplary embodiment, there may be two xylene fractionation column with a bottoms of a first xylene fractionation column being passed to a second xylene fractionation column located downstream of the first xylene fractionation column. In such an embodiment, reformate bottoms stream in line 108 and the BT column bottoms stream in line 166 may be provided to the first xylene fractionation column and the deheptanizer bottoms stream in line 148 may be passed to the second xylene fractionation column. In accordance with an exemplary embodiment as shown in the FIG. 1, an overhead xylene stream in line 112 is withdrawn from the xylene fractionation column 110. The overhead xylene stream may include the portion of the reformate bottoms stream. Further, a xylene fractionator bottoms stream in line 114 rich in C₉ and heavier alkylaromatic hydrocarbons is withdrawn.

In accordance with an exemplary embodiment as shown, the overhead xylene stream may be recovered from an overhead of the xylene fractionation column 110 after condensing, flashing and refluxing a portion of the overhead stream from the column. Further, as illustrated, the xylene fractionation column 110 may include a heater at a bottom of the column to vaporize and send a portion of the bottoms stream back to the bottom of the column.

The overhead xylene stream in line 112 includes para-xylene, meta-xylene, ortho-xylene and ethylbenzene and may be subsequently passed to the para-xylene separation unit 116 to obtain a desired C₈ aromatic isomer product via a separation process. In accordance with an exemplary embodiment as shown in FIG. 1, there is no intermediate olefin treatment step between the xylene fractionation column 110 and para-xylene separation unit 116. In the para-xylene separation unit 116, the overhead xylene stream is contacted with an adsorbent under adsorption conditions. In an embodiment, the adsorption conditions may include an adsorption temperature of less than about 175° C. (350° F.). In accordance with an exemplary embodiment, the adsorbent may be a binderless adsorbent comprising zeolite X having an average crystallite size of less than 1.8 microns. In another aspect, the average crystallite size is from about 500 nanometers to about 1.5 microns. In an aspect, the adsorbent may have at least 95% of its ion-exchangeable sites exchanged with barium or a combination of barium and potassium. In accordance with an exemplary embodiment, the binderless adsorbent may include a converted portion of zeolite X resulting from the conversion of a zeolite X-precursor. Examples of the zeolite X-precursor include, but are not limited to, kaolin clay. Binderless catalysts that can be used in the present disclosure include conventional binderless zeolite catalysts such as those disclosed in U.S. Pat. No. 8,283,274, U.S. Pat. No. 7,812,208, U.S. Pat. No. 7,820,869, and U.S. Pat. Publ. No. 20090326308, the teachings of which are incorporated herein by reference. Applicants have found that such binderless adsorbents are unaffected with respect to capacity and selectivity when subjected to olefins. The Bromine Index of the inlet and outlet of the para-xylene separation unit remains unchanged, indicating that olefins pass through the adsorbent. Accordingly, in various embodiments, a typical olefin treatment unit present between the reformate splitter column 104 and the para-xylene separation unit 116 may be removed. In an aspect, a typical olefin treatment unit that is placed downstream of the reformate splitter bottoms may also be removed.

In accordance with the instant embodiment as discussed, the separation process in the para-xylene separation unit 116 operates, preferably via simulated moving adsorption bed (SMB) employing a desorbent, to provide a para-xylene extract stream in line 118 comprising a mixture of para-xylene and desorbent for the instant embodiment. Examples of desorbent include, and are not limited to para-diethylbenzene. The para-xylene extract stream in line 118 may be passed to the extract column 120 which separates para-xylene from the desorbent. A para-xylene stream in line 122 may be withdrawn comprising the desired para-xylenes from the extract column 120. Further, a first return desorbent stream in line 124 is withdrawn which may be subsequently recycled to the para-xylene separation unit 116. The para-xylene stream in line 122 may be passed to the finishing column 126 to separate a para-xylene product in line 130 from the lighter hydrocarbons obtained as an finishing column overhead stream in line 128. In accordance with an exemplary embodiment as shown in the FIG. 1, the finishing column overhead stream may be passed to the BT column 160.

A raffinate stream in line 119 comprising non-equilibrium mixture of C₈ aromatics raffinate and the desorbent may be also withdrawn from the para-xylene separation unit 116. In accordance with an exemplary embodiment as shown in the FIG. 1, the raffinate stream in line 119 may be passed to the raffinate column 132. The raffinate column 132 separates a raffinate product stream in line 134 for isomerization in the isomerization unit 140 from a second return desorbent stream in line 136. In embodiments, there can be more than one raffinate columns. In accordance with an exemplary embodiment there may be two raffinate columns with a bottoms of a first raffinate column being passed to a second raffinate column located downstream of the first raffinate column. In accordance with an exemplary embodiment as shown in FIG. 1, the first desorbent rerun stream in line 124 and the second desorbent rerun stream in line 136 may combine to provide a combined desorbent rerun stream in line 138 which may be subsequently passed to the para-xylene separation unit 116.

The raffinate product stream in line 134 comprising a non-equilibrium mixture of xylene isomers and ethylbenzene is introduced to the isomerization unit 140 to provide an isomerization effluent in line 142. The raffinate product stream is isomerized in reactor 152, which contains an isomerization catalyst to provide a product approaching equilibrium concentrations of C₈ aromatic isomers. In accordance with the instant embodiment as discussed for producing para-xylenes, additional para-xylene may be produced by reestablishing an equilibrium or near-equilibrium distribution of xylene isomers. In accordance with an exemplary embodiment as discussed, the isomerization catalyst is an ethylbenzene (hereinafter “EB”) dealkylation catalyst. In the isomerization unit 140, the non-equilibrium mixture, depleted in para-xylene, is contacted with an EB dealkyl type catalyst well-known in the art. The isomerization catalyst favorably comprises a zeolitic aluminosilicate selected from those which have a Si:Al₂ ratio greater than about 10, preferably greater than 20, and a pore diameter of about 5 to 8 angstroms (Å). Specific examples of suitable zeolites are the MFI, MEL, EUO, FER, MFS, MTT, MTW, TON, MOR and FAU types of zeolites. A particularly favored MFI-type zeolite is gallium-MFI, with gallium as a component of the crystal structure.

The isomerization effluent is withdrawn in line 142 from the isomerization unit 140. The isomerization effluent from isomerization unit may be passed to the deheptanizer column 144. The deheptanizer bottoms stream in line 148 from the deheptanizer column 144 contains C₈ aromatics including para-xylene. In the instant embodiment using EB Dealkylation type catalyst, olefin saturation is typically not required. Accordingly, as shown in the FIG. 1, the deheptanizer bottoms stream in line 148 may be recycled to the xylene fractionation column 110 and processed further as previously described. A deheptanizer overhead stream in line 146 may also be withdrawn from the deheptanizer column 144 and may be passed to the stabilizer 178.

Referring back to the reformate splitter column 104, the reformate overhead stream in line 106 comprising C₇₋ aromatic hydrocarbons may be passed to the aromatics extraction unit 150. The aromatics extraction unit 150 can comprise different methods of separating aromatics from a hydrocarbon stream. One industry standard is the Sulfolane™ process, which is an extractive distillation process utilizing sulfolane to facilitate high purity extraction of aromatics. The Sulfolane™ process is well known to those skilled in the art. An aromatics extract stream in line 154 comprising benzene and toluene and a raffinate stream in line 152 comprising non-aromatic hydrocarbons may be withdrawn from the aromatics extraction unit 150. The aromatics extract stream in line 154 may be passed to the BT column 160 to provide benzene and toluene via separation. In accordance with an exemplary embodiment as shown in FIG. 1, the aromatics extract stream in line 154 may be passed through a clay treater 156 to treat residual olefin contaminants and provide a treated aromatics extract stream in line 158 prior to being passed to the BT column 160. A transalkylation bottom stream in line 176 from the transalkylation stripper column 172 may also be passed to the BT column 160. A benzene-enriched stream in line 162 and a toluene-enriched stream in line 164 are withdrawn from the BT column 160. Further, the BT column bottoms stream in line 166 is withdrawn and sent to the xylene fractionation column 110 for further processing as described above. The toluene-enriched stream in line 164 may be passed to the transalkylation unit 168 for production of additional xylenes and benzene.

In accordance with an exemplary embodiment as shown in FIG. 1, in addition to toluene-enriched stream, a heavy aromatics column overhead stream in line 186 rich in C₉ and C₁₀ alkylaromatics from the heavy aromatics column 184 may be passed to the transalkylation unit 168. A make-up hydrogen gas stream (not shown) may also be provided to the transalkylation unit 168. In the transalkylation unit 168, the incoming feedstreams may be contacted with a transalkylation catalyst under transalkylation conditions. In the transalkylation unit 168, the process continues by transalkylating C₉ and C₁₀ alkylaromatics with toluene. A transalkylated stream in line 170 comprising benzene and xylenes may be withdrawn from the transalkylation unit 168.

Transalkylation catalysts that can be used in the present disclosure include conventional transkylation catalysts such as those disclosed in U.S. Pat. No. 6,740,788, the teachings of which are incorporated herein by reference. Conditions employed in the transalkylation unit 200 normally include a temperature of from about 200° C. to about 540° C. The transalkylation unit 200 is operated at moderately elevated pressures broadly ranging from about 1 kg/cm′ to about 60 kg/cm². The transalkylation reaction can be effected over a wide range of space velocities, with higher space velocities affecting a higher ratio of para-xylene at the expense of conversion. Liquid hourly space velocity generally is in the range of from about 0.1 to about 20 hr⁻¹.

The transalkylated stream in line 170 may be sent to transalkylation stripper 172 to recover the transalkylation stripper bottoms stream in line 176. A net overhead stream in line 174 comprising C₆ and lighter hydrocarbons may also be withdrawn from the transalkylation stripper 174. Subsequently, the transalkylation stripper bottoms stream in line 176 may be recycled to the BT column 160 to recover benzene product and unconverted toluene for further processing as previously described. The net overhead stream in line 174 may be passed to the stabilizer 178 to provide a stabilizer overhead vaporous stream in line 180 and a stabilizer bottoms stream in line 182. The stabilizer overhead vaporous stream in line 180 may be recycled to the transalkylation stripper 172 as shown in the FIG. 1. The stabilizer bottoms stream in line 182 may be passed to the aromatics extraction unit 180.

Referring back to the xylene fractionation column 110, the xylene fractionator bottoms stream in line 114 rich in C₉ and heavier alkylaromatic hydrocarbons is passed to the heavy aromatics column 184 to separate heavy aromatics comprising C₁₁₊ alkylaromatic hydrocarbons from C₉ and C₁₀ alkylaromatics recovered as the heavy aromatics column overhead stream in line 186. The C₁₁₊ alkylaromatic hydrocarbons may be withdrawn from the heavy aromatics column 184 as a bottoms stream in line 188. The heavy aromatics column overhead stream in line 186 rich in C₉ and C₁₀ alkylaromatics may be passed to the transalkylation unit 164 for production of additional xylenes and benzene as previously described.

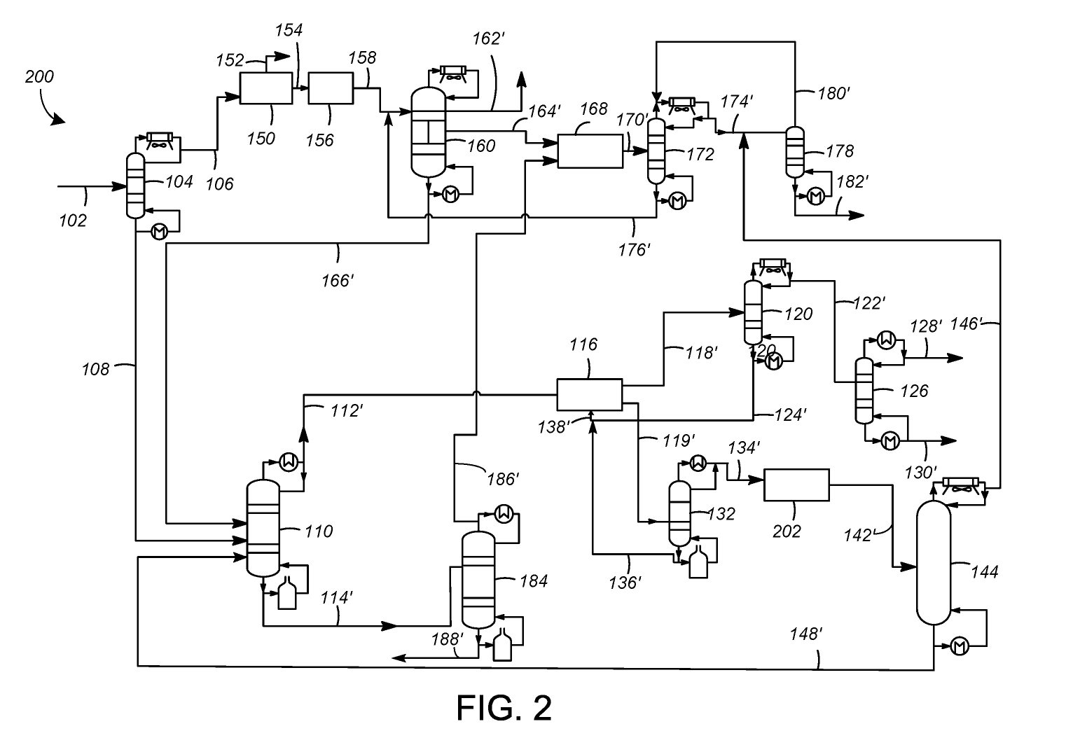

Turning now to FIG. 2, another embodiment of the aromatics complex is addressed with reference to a process and apparatus 200 for producing a para-xylene product in an aromatic complex is addressed. Many of the elements in FIG. 2 have the same configuration as in FIG. 1 and bear the same respective reference number and have similar operating conditions. Further, streams that have same configuration as in FIG. 1 but vary in composition bear the same reference numeral as in FIG. 1 but are marked with a prime symbol (′). The apparatus and process in FIG. 2 are the same as in FIG. 1 with the exception of the noted following differences. The apparatus 200 includes an isomerization unit 202 including an ethylbenzene (hereinafter “EB”) isomerization type catalyst in contrast of EB dealkylation catalyst of FIG. 1. In the isomerization unit 202, the non-equilibrium mixture, depleted in para-xylene, is contacted with an EB Isom type catalyst well-known in the art. An isomerization effluent is withdrawn in line 142′ from the isomerization unit 140′. The isomerization effluent from isomerization unit may be passed to the deheptanizer column 144. A deheptanizer bottoms stream in line 148′ may be withdrawn from the deheptanizer column 144 contains C₈ aromatics including para-xylene. Typically, in processes using EB Isom type catalyst, the deheptanizer bottoms stream is passed passed through an olefin treatment unit to treat residual olefin contaminants before being passed to the xylene fractionation column 110. Examples of the olefin treatment unit include, but are not limited to, a clay treater and an olefin reduction process (ORP) unit. In accordance with an exemplary embodiment as shown in FIG. 2, the deheptanizer bottoms stream in line 148′ may be passed directly to the xylene fractionation column 110 without an intermediate step for removal of olefins. Accordingly, the xylene fractionation column 110 may be in direct, downstream communication with the deheptanizer column 144. Accordingly, a portion of the deheptanizer bottoms stream may be passed to the para-xylene separation unit 116 without an intermediate step for removal of olefins. Rest of process flow is similar to as described in FIG. 1.

### Specific Embodiments

While the following is described in conjunction with specific embodiments, it will be understood that this description is intended to illustrate and not limit the scope of the preceding description and the appended claims.

A first embodiment of the invention is a process for the production of para-xylene, wherein the process comprises a) providing a reformate stream comprising aromatic hydrocarbons to a reformate splitter to provide a reformate bottoms stream comprising C7+ aromatic hydrocarbons and a reformate overhead stream comprising C7− aromatic hydrocarbons; and b) passing a portion of the reformate bottoms stream to a para-xylene separation unit for separating para-xylene, wherein the portion is contacted with an adsorbent under adsorption conditions to provide a xylene extract stream comprising para-xylene and a raffinate product stream, wherein the portion of the reformate bottoms stream is passed to the para-xyelene separation unit without an intermediate step for removal of olefins. An embodiment of the invention is one, any or all of prior embodiments in this paragraph up through the first embodiment in this paragraph further comprising passing the reformate bottoms stream to a xylene fractionation column without the intermediate step for removal of olefins to produce a xylene fractionator bottoms stream rich in C9 and heavier alkylaromatic hydrocarbons and an overhead xylene stream comprising the portion of the reformate bottoms stream. An embodiment of the invention is one, any or all of prior embodiments in this paragraph up through the first embodiment in this paragraph, wherein the intermediate step comprises a clay treater. An embodiment of the invention is one, any or all of prior embodiments in this paragraph up through the first embodiment in this paragraph, wherein the intermediate step comprises an olefin reduction process (ORP) unit. An embodiment of the invention is one, any or all of prior embodiments in this paragraph up through the first embodiment in this paragraph, wherein the the adsorbent is a binderless adsorbent comprising zeolite X having an average crystallite size of less than 1.8 microns. An embodiment of the invention is one, any or all of prior embodiments in this paragraph up through the first embodiment in this paragraph, wherein the adsorbent has at least 95% of its ion-exchangeable sites exchanged with barium or a combination of barium and potassium. An embodiment of the invention is one, any or all of prior embodiments in this paragraph up through the first embodiment in this paragraph, wherein the para-xylene separation unit is a simulated moving bed adsorption unit.

A second embodiment of the invention is a process for the production of para-xylene, wherein the process comprises a) introducing a raffinate product stream comprising C8 aromatic isomers to an isomerization unit to provide an isomerization effluent, wherein the isomerization effluent is produced in the presence of an ethylbenzene (EB) isomerization catalyst; b) passing the isomerization effluent to a deheptanizer column to provide a deheptanizer bottoms stream comprising C7+ aromatics; and c) passing a portion of the deheptanizer bottoms stream to a para-xylene separation unit for separating para-xylene, wherein the portion is contacted with an adsorbent under adsorption conditions to provide a xylene extract stream comprising para-xylene and the raffinate product stream, wherein the portion of the deheptanizer bottoms stream is passed to the para-xyelene separation unit without an intermediate step for removal of olefins. An embodiment of the invention is one, any or all of prior embodiments in this paragraph up through the second embodiment in this paragraph further comprising passing the deheptanizer bottoms stream to a xylene fractionation column without the intermediate step for removal of olefins, to provide an overhead xylene stream comprising the portion of the deheptanizer bottoms stream. An embodiment of the invention is one, any or all of prior embodiments in this paragraph up through the second embodiment in this paragraph, wherein the intermediate step comprises a clay treater. An embodiment of the invention is one, any or all of prior embodiments in this paragraph up through the second embodiment in this paragraph, wherein the intermediate step comprises an olefin reduction process (ORP) unit. An embodiment of the invention is one, any or all of prior embodiments in this paragraph up through the second embodiment in this paragraph, wherein the adsorbent is a binderless adsorbent comprising zeolite X having an average crystallite size of less than 1.8 microns. An embodiment of the invention is one, any or all of prior embodiments in this paragraph up through the second embodiment in this paragraph, wherein the adsorbent has at least 95% of its ion-exchangeable sites exchanged with barium or a combination of barium and potassium. An embodiment of the invention is one, any or all of prior embodiments in this paragraph up through the second embodiment in this paragraph, wherein the para-xylene separation unit is a simulated moving bed adsorption unit.

A third embodiment of the invention is a process for the production of para-xylene, wherein the process comprises a) providing a reformate stream comprising aromatic hydrocarbons to a reformate splitter to provide a reformate bottoms stream comprising C7+ aromatic hydrocarbons and a reformate overhead stream comprising C7− aromatic hydrocarbons; b) introducing a raffinate product stream comprising C8 aromatic isomers to an isomerization unit to provide an isomerization effluent, wherein the isomerization effluent is produced in the presence of an ethylbenzene (EB) isomerization catalyst; c) passing the isomerization effluent to a deheptanizer column to provide a deheptanizer bottoms stream comprising C7+ aromatics; and d) passing a portion of the reformate bottoms stream and a portion of the deheptanizer bottoms stream to a para-xylene separation unit for separating para-xylene, wherein the reformate bottom portion and the deheptanizer bottom portion are contacted with an adsorbent under adsorption conditions to provide a xylene extract stream comprising para-xylene and the raffinate product stream, wherein the portion of the isomerization effluent and the portion of the deheptanizer bottoms stream is passed to the para-xyelene separation unit without an intermediate step for removal of olefins. An embodiment of the invention is one, any or all of prior embodiments in this paragraph up through the third embodiment in this paragraph, wherein the intermediate step comprises a clay treater. An embodiment of the invention is one, any or all of prior embodiments in this paragraph up through the third embodiment in this paragraph, wherein the intermediate step comprises an olefin reduction process (ORP) unit. An embodiment of the invention is one, any or all of prior embodiments in this paragraph up through the third embodiment in this paragraph, wherein the adsorbent is a binderless adsorbent comprising zeolite X having an average crystallite size of less than 1.8 microns. An embodiment of the invention is one, any or all of prior embodiments in this paragraph up through the third embodiment in this paragraph, wherein the adsorbent has at least 95% of its ion-exchangeable sites exchanged with barium or a combination of barium and potassium. An embodiment of the invention is one, any or all of prior embodiments in this paragraph up through the third embodiment in this paragraph, wherein the para-xylene separation unit is a simulated moving bed adsorption unit.

Without further elaboration, it is believed that using the preceding description that one skilled in the art can utilize the present invention to its fullest extent and easily ascertain the essential characteristics of this invention, without departing from the spirit and scope thereof, to make various changes and modifications of the invention and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

1. A process for the production of para-xylene, wherein the process comprises:
a) providing a reformate stream comprising aromatic hydrocarbons to a reformate splitter to provide a reformate bottoms stream comprising C₇₊ aromatic hydrocarbons and a reformate overhead stream comprising C₇₋ aromatic hydrocarbons; and
b) passing a portion of the reformate bottoms stream to a para-xylene separation unit for separating para-xylene, wherein said portion is contacted with an adsorbent under adsorption conditions to provide a xylene extract stream comprising para-xylene and a raffinate product stream,
wherein the portion of the reformate bottoms stream is passed to the para-xyelene separation unit without an intermediate step for removal of olefins.

2. The process of claim 1 further comprising passing the reformate bottoms stream to a xylene fractionation column without the intermediate step for removal of olefins to produce a xylene fractionator bottoms stream rich in C₉ and heavier alkylaromatic hydrocarbons and an overhead xylene stream comprising the portion of the reformate bottoms stream.

3. The process of claim 1, wherein the intermediate step comprises a clay treater.

4. The process of claim 1, wherein the intermediate step comprises an olefin reduction process (ORP) unit.

5. The process of claim 1, wherein the said adsorbent is a binderless adsorbent comprising zeolite X having an average crystallite size of less than 1.8 microns.

6. The process of claim 5, wherein the adsorbent has at least 95% of its ion-exchangeable sites exchanged with barium or a combination of barium and potassium.

7. The process of claim 1, wherein the para-xylene separation unit is a simulated moving bed adsorption unit.

8. A process for the production of para-xylene, wherein the process comprises:
a) introducing a raffinate product stream comprising C₈ aromatic isomers to an isomerization unit to provide an isomerization effluent, wherein the isomerization effluent is produced in the presence of an ethylbenzene (EB) isomerization catalyst;
b) passing the isomerization effluent to a deheptanizer column to provide a deheptanizer bottoms stream comprising C7+ aromatics; and
c) passing a portion of the deheptanizer bottoms stream to a para-xylene separation unit for separating para-xylene, wherein said portion is contacted with an adsorbent under adsorption conditions to provide a xylene extract stream comprising para-xylene and the raffinate product stream,
wherein the portion of the deheptanizer bottoms stream is passed to the para-xyelene separation unit without an intermediate step for removal of olefins.

9. The process of claim 8 further comprising passing the deheptanizer bottoms stream to a xylene fractionation column without the intermediate step for removal of olefins, to provide an overhead xylene stream comprising the portion of the deheptanizer bottoms stream.

10. The process of claim 8, wherein the intermediate step comprises a clay treater.

11. The process of claim 8, wherein the intermediate step comprises an olefin reduction process (ORP) unit.

12. The process of claim 8, wherein the adsorbent is a binderless adsorbent comprising zeolite X having an average crystallite size of less than 1.8 microns.

13. The process of claim 8, wherein the adsorbent has at least 95% of its ion-exchangeable sites exchanged with barium or a combination of barium and potassium.

14. The process of claim 8, wherein the para-xylene separation unit is a simulated moving bed adsorption unit.

15. A process for the production of para-xylene, wherein the process comprises:
a) providing a reformate stream comprising aromatic hydrocarbons to a reformate splitter to provide a reformate bottoms stream comprising C₇₊ aromatic hydrocarbons and a reformate overhead stream comprising C₇₋ aromatic hydrocarbons;
b) introducing a raffinate product stream comprising C₈ aromatic isomers to an isomerization unit to provide an isomerization effluent, wherein the isomerization effluent is produced in the presence of an ethylbenzene (EB) isomerization catalyst;
c) passing the isomerization effluent to a deheptanizer column to provide a deheptanizer bottoms stream comprising C7+ aromatics; and
d) passing a portion of the reformate bottoms stream and a portion of the deheptanizer bottoms stream to a para-xylene separation unit for separating para-xylene, wherein the reformate bottom portion and the deheptanizer bottom portion are contacted with an adsorbent under adsorption conditions to provide a xylene extract stream comprising para-xylene and the raffinate product stream,
wherein the portion of the isomerization effluent and the portion of the deheptanizer bottoms stream is passed to the para-xyelene separation unit without an intermediate step for removal of olefins.

16. The process of claim 15, wherein the intermediate step comprises a clay treater.

17. The process of claim 15, wherein the intermediate step comprises an olefin reduction process (ORP) unit.

18. The process of claim 15, wherein the adsorbent is a binderless adsorbent comprising zeolite X having an average crystallite size of less than 1.8 microns.

19. The process of claim 18, wherein the adsorbent has at least 95% of its ion-exchangeable sites exchanged with barium or a combination of barium and potassium.

20. The process of claim 15, wherein the para-xylene separation unit is a simulated moving bed adsorption unit.
